(19) United States
(12) Patent Application Publication
(10) Pub. No.: US 2026/0007617 A1
(43) Pub. Date: Jan. 8, 2026

(54) METHOD FOR TREATING RENAL CELL CARCINOMA OR CANCER

(71) Applicant: Lantern Pharma Inc., Dallas, TX (US)

(72) Inventors: Jianli Zhou, Dallas, TX (US); Kishor Bhatia, Atlanta, GA (US)

(73) Assignee: Lantern Pharma Inc., Dallas, TX (US)

(21) Appl. No.: 19/322,966

(22) Filed: Sep. 9, 2025

Related U.S. Application Data
(63) Continuation of application No. PCT/US2024/019456, filed on Mar. 11, 2024.
(60) Provisional application No. 63/451,159, filed on Mar. 9, 2023.

(51) Int. Cl. A61K 31/122; A61K 45/06; A61P 35/04

(52) U.S. Cl. CPC A61K 31/122; A61K 45/06; A61P 35/04

(57) ABSTRACT

This method involves treating individuals diagnosed with renal cell carcinoma using a therapeutically effective amount of hydroxyureamethyl acylfulvene. The treatment is applicable to various types of renal cell carcinoma, including clear cell renal cell carcinoma, papillary renal cell carcinoma, chromophobe renal cell carcinoma, collecting duct carcinoma, and medullary carcinoma.

### CROSS-REFERENCE TO RELATED APPLICATION

This is application a continuation of International Patent Application No. PCT/US2024/019456, filed on Mar. 11, 2024, which claims the benefit of U.S. Provisional Patent Application No. 63/451,159, filed Mar. 9, 2023, each of which is incorporated by reference herein in its entirety.

### TECHNICAL FIELD

This application relates to treatment of renal cell carcinoma or cancer and more particularly this application to compositions comprising acylfulvene and methods of using the acylfulvene to treat kidney cancer.

### BACKGROUND

Renal cancer, also recognized as one of the top ten most common cancers globally, impacts approximately 1 in every 63 individuals throughout their lifetime. Predominantly, this type of cancer affects adults aged between 50 and 80 years. A notable trend has been observed where North America currently reports the highest incidence rates of renal cancer. However, in developing countries, there has been a consistent and alarming rise in cases over the past thirty years, highlighting the increasing global burden of this disease.

Renal cell cancer, alternatively known as kidney cancer or renal cell adenocarcinoma, is characterized by the growth of malignant (cancerous) cells within the lining of the tubules, which are minuscule tubes within the kidneys. The kidneys, located on each side of the spine and above the waist, function as the body's natural filtration system. They filter the blood, removing waste products and converting them to urine. This urine then travels from each kidney through a tube called a ureter to the bladder, where it is stored until it exits the body through the urethra. The most prevalent form of kidney cancer is renal cell carcinoma (RCC), which primarily affects the microscopic tubules within these organs.

The treatment of renal cell carcinoma represents a significant challenge in the medical field. It is known as one of the most arduous malignancies to manage effectively due to the limited availability of effective therapies. Currently, biological therapy provides some degree of benefit, albeit minimal, for patients suffering from RCC. The substantial drug resistance often associated with RCC complicates treatment further; the exact mechanisms behind this resistance remain unclear, though theories suggest a role for the expression of certain membrane transporters and altered metabolism. Despite ongoing research, there has been a shortage of anticancer drugs developed specifically targeting RCC, underscoring the critical need for innovative therapeutic approaches and research in this area.

Accordingly, there is always a need for improved methods to treat kidney cancer.

### SUMMARY

This application discloses the discovery that treatment of a kidney or renal cancer in a subject with acylfulvene. In one aspect, this application provides a method for treating kidney cancer by administering a therapeutically effective amount of acylfulvene, or a pharmaceutically acceptable salt thereof, to an afflicted subject.

Another aspect includes a therapy that involves the administration of a combination of active agents, including illudin or an illudin analog such as acylfulvene.

Another aspect of provides pharmaceutical compositions comprising an illudin or illudin analog (e.g., acylfulvene) or pharmaceutically acceptable salts thereof, mixed with pharmaceutically suitable carriers or excipient(s) at doses to treat or prevent kidney cancer. The pharmaceutical compositions can also be administered in combination with other therapeutic agents or therapeutic modalities simultaneously, sequentially, or in alternation.

An aspect provides a method for treating a subject, specifically a human cancer patient, who requires acylfulvene treatment. This includes patients whose kidney cancer has become refractory to standard chemotherapeutic regimens or whose cancer has relapsed following standard chemotherapy. This encompasses renal cancers such as metastatic cell carcinoma, Wilms' tumor (nephroblastoma), renal sarcoma, and various benign (non-cancerous) renal tumors like renal adenoma, neoplastic cytoma, and angiomyolipoma, with a particular emphasis on clear cell renal cell carcinoma.

Another aspect includes a therapy or method of treatment for renal cancers that are either resistant to standard therapy or are metastatic, covering a range of conditions from clear cell carcinoma of the kidney, metastatic cell carcinoma, Wilms' tumor (nephroblastoma), renal sarcoma, to benign renal tumors, including renal adenoma, tumor cell tumors, and angiomyolipoma.

Another aspect includes the therapy in which the acylfulvene is (−)-hydroxyureamethyl acylfulvene.

Another aspect includes a method for treating kidney cancer in a subject in need thereof comprising administering to the subject an effective amount of hydroxyureamethyl acylfulvene. The hydroxyureamethyl acylfulvene can be (−)-hydroxyureamethyl acylfulvene or hydroxyureamethyl acylfulvene is (+)-hydroxyureamethyl acylfulvene. The hydroxyureamethyl acylfulvene can be administered in a dosage range of 0.1 to 100 mg/kg of body weight per day. The kidney cancer can be clear cell renal carcinoma, papillary renal cell carcinoma, chromophobe renal cell carcinoma, and collecting duct carcinoma.

Another aspect includes a pharmaceutical composition comprising hydroxyureamethyl acylfulvene and a pharmaceutically acceptable carrier, for use in treating kidney cancer. The hydroxyureamethyl acylfulvene can be administered in combination with at least one other anticancer agent.

Another aspect includes a treatment that leads to a decrease in tumor size or progression.

Another aspect of this application includes the treatments of cancer that can include solid tumors, and hematological malignances.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 illustrates the application of LP-184 on various renal cell lines, including both epithelial and carcinoma types;

FIG. 2 demonstrates that cells containing 6-4 photoproducts (6-4PPs) were significantly eradicated after 5 and 7 hours;

FIG. 3 presents a western blot analysis conducted on the cell lines previously investigated; and

FIG. 4 displays the sensitivity of various cell lines to LP-184, noting a significant reduction in cell viability over 72 hours.

### DETAILED DESCRIPTION

This application provides therapeutic methods for treating renal cell carcinoma or cancer in subjects. One embodiment describes a method for treating subject with renal cell carcinoma, which includes the administration of an effective amount of acylfulvene (for example, hydroxyureamethyl acylfulvene) or its salt. In other embodiments, the therapy comprises administering a combination of various treatments. One embodiment specifically can include Renal Cell Carcinoma (RCC). The renal cell carcinoma can include clear cell renal cell carcinoma, papillary renal cell carcinoma, chromophobe renal cell carcinoma, and other rare types of renal cell carcinoma (such as collecting duct RCC, multicystic RCC, medullary carcinoma, mucinous tubular and spindle cell carcinoma, neuroblastoma-associated RCC, and unclassified renal cell carcinoma), as well as metastatic RCC. In another embodiment, kidney cancer is defined to include metastatic cell carcinoma, Wilms' tumor (nephroblastoma), renal sarcoma, and benign (non-cancerous) renal tumors such as renal adenoma, neoplastic cytoma, and angiomyolipoma.

### Illudin or Acylfulvene

In one embodiment, this application discusses the use of an illudin or an illudin analog, such as acylfulvene. Acylfulvene represents a class of cytotoxic, semi-synthetic derivatives originating from illudin, a natural compound extracted from the jack o′lantern mushroom (Omphalotus olearius). Acylfulvene is created from the sesquiterpene illudin S through an acid treatment (reverse Prins reaction) and exhibits significantly lower reactivity to thiols compared to illudin S.

As an example, acylfulvene, specifically (−)-hydroxyureamethyl acylfulvene (referred to as LP-184 by Lantern Pharma Inc.), which induces a positive shift in light, is illustrated below:

In another example, the acylfulvene used is (+)-hydroxyureamethyl acylfulvene (referred to as LP-284 by Lantern Pharma Inc.), which induces a negative shift in light, as illustrated below:

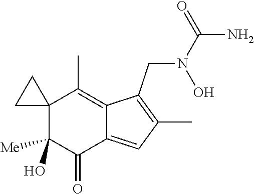 [SMILES: CC1=C(CN(O)C(N)=O)C2=C(C)C3(CC3)[C@](C)(O)C(=O)C2=C1]

- - acylfulvene are enantiomers and are now publicly known.

In another example, the acylfulvene discussed is Irofulven.

In one embodiment, the patient is treated with radiation prior to, concurrently with, and/or after treatment with an acylfulvene.

In one embodiment, the method includes the administration of acylfulvene to treat kidney cancer. As used herein, the term “kidney cancer” refers to a cancer originating from the kidney, as commonly understood in the medical field. In some embodiments, the kidney cancer is renal cell carcinoma (RCC). The terms “renal cell cancer” and “renal cell carcinoma” refer to cancer that originates in the lining of the proximal convoluted tubule. Specifically, RCC includes several common histologic subtypes: clear cell renal cell carcinoma, papillary renal cell carcinoma, chromophobe renal cell carcinoma, collecting duct carcinoma, and medullary carcinoma, with clear cell renal cell carcinoma (ccRCC) being the most common subtype.

In another embodiment, a method of treating conventional or clear cell renal cell carcinoma is provided. The cells can be abnormal cells. They can be studied in vitro or in vivo. In certain embodiments, the cell is a proliferative cell or a stem cell.

In another embodiment, a second therapeutic may consist of one or more chemotherapeutic agents selected from camptothecin derivatives, paclitaxel, docetaxel, epothilone B, 5-FU, gemcitabine, oxaliplatin, cisplatinum, carboplatin, melphalan, dacarbazine, temozolomide, doxorubicin, imatinib, erlotinib, bevacizumab, cetuximab, and a Raf kinase inhibitor.

In another embodiment, the second therapeutic comprises one or more chemotherapeutic agents selected from paclitaxel and cisplatinum.

Another embodiment includes a pharmaceutical composition containing a therapeutically effective amount of an illudin or an illudin analog thereof, derivative, or a pharmaceutically acceptable salt thereof; and a therapeutically effective amount of spironolactone or an analog, derivative, or a pharmaceutically acceptable salt thereof. The illudin analog could be hydroxyureamethyl acylfulvene.

In another embodiment, a kit for the treatment of cancer in a subject includes a therapeutically effective amount of an illudin or an illudin analog thereof, derivative, or a pharmaceutically acceptable salt thereof; and a therapeutically effective amount of spironolactone or an analog, derivative, or a pharmaceutically acceptable salt thereof.

In another embodiment, the second therapeutic comprises one or more chemotherapeutic agents selected from camptothecin derivatives, paclitaxel, docetaxel, epothilone B, 5-FU, gemcitabine, oxaliplatin, cisplatinum, carboplatin, melphalan, dacarbazine, temozolomide, doxorubicin, imatinib, erlotinib, bevacizumab, cetuximab, and a Raf kinase inhibitor.

In another embodiment, the second therapeutic includes one or more chemotherapeutic agents selected from paclitaxel or cisplatinum.

In one embodiment, expression or elevated expression of the ACTB gene or protein indicates a more effective treatment with LP-184 or (−)-hydroxyureamethyl acylfulvene. The ACTB gene encodes the β-actin protein, which is a part of the actin protein family. This family forms an organized network known as the actin cytoskeleton, which provides structural support within cells. Among the six distinct types of actin, four are specific to muscle cells, aiding in muscle contraction, while the remaining two, β-actin and γ-actin—originating from the ACTG1 gene—are ubiquitously present in various cell types throughout the body. In renal cancer treatment where ACTB (beta-actin) measurement is involved, the “predetermined level” can be a specific concentration of ACTB in the patient's tissue or blood samples.

The term “combination therapy” includes the administration of therapeutic agents described above in combination with other biologically active ingredients and non-drug therapies (e.g., surgery or radiation treatment). If the combination therapy comprises a non-drug treatment, this treatment can occur at any suitable time, provided there is a beneficial effect from the combination of therapeutic agents and non-drug treatment. For instance, beneficial effects can still be achieved even if non-drug treatments are temporally separated from the administration of therapeutic agents, potentially by days or even weeks.

In another aspect, a composition or combination therapy, or a pharmaceutically acceptable salt or solvate thereof, may be administered in combination with radiation therapy. Radiation therapy can be administered together with a composition from the current invention and another chemotherapeutic agent as part of a multi-agent therapy.

Combination therapy may involve administering two or more agents, such as an acylfulvene, a spironolactone, and one or more other therapeutic agents, each formulated and administered separately, or by administering multiple agents in a single formulation. Various combinations are possible under combination therapy. For example, two agents can be combined in one formulation and administered alongside a separate formulation containing a third agent. While these agents can be administered simultaneously, they can also be administered sequentially, with intervals ranging from minutes to weeks between them.

Therapeutically effective doses may vary depending on the condition treated, severity, administration route, patient age and health, excipient use, potential concurrent therapies, and the physician's judgment. Effective doses can be guided by the prescribing information for hydroxyureamethyl acylfulvene or related medical literature.

The term “effective amount” refers to a quantity of an agent sufficient to alleviate one or more symptoms of a disease or disorder. A “therapeutically effective amount” is an amount adequate to provide a desired effect in a typical patient. An “effective amount” may delay symptom development, alter disease course, or reverse disease symptoms. The precise “effective amount” can vary and should be determined by medical professionals through routine experimentation.

According to the methods described herein, a “subject in need thereof” is a subject diagnosed with kidney cancer, at increased risk of kidney cancer compared to the general population, or showing no significant response to existing cancer therapies. In this context, “non-responsive” or “refractory” denotes insufficient clinical improvement after treatment. In some scenarios, the subject in need thereof is a cancer patient whose disease is refractory to standard therapy or recurs after standard therapy.

The dosage ranges for administering an agent, as described herein, depend upon factors including the agent's form, potency, and the desired extent of symptom, marker, or condition indicator reduction, such as the targeted percentage reduction in tumor growth. The dosage should not be so large as to cause adverse side effects. Typically, the dosage will vary with the patient's age, condition, and sex, and can be determined by a skilled professional. Furthermore, the dosage can be adjusted by the individual physician should complications arise.

The term “therapeutically effective amount” refers to a quantity of a pharmaceutical agent that is sufficient to treat, ameliorate, or prevent an identified disease or condition, or to manifest a detectable therapeutic or inhibitory effect. This effect can be assessed by any known assay method. The precise amount needed will depend on factors such as the subject's body weight, size, and health; the nature and extent of the condition; and the chosen therapy or combination thereof. A clinician can determine therapeutically effective amounts through routine experimentation, based on their skill and judgment. Preferably, the condition to be treated is cancer, but it may also be a cell proliferative disorder.

The efficacy of an agent, in the treatment of conditions described herein or in inducing a described response (e.g., for solid), is assessable by a skilled clinician. A treatment is deemed “effective” if it beneficially alters one or more signs or symptoms of the condition, improves other clinically accepted symptoms, or induces a desired response by at least 10% following the described methods. Efficacy can be evaluated by measuring markers, indicators, symptoms, incidences of the treated condition, or any other quantifiable parameters such as tumor size and/or growth rate. Moreover, efficacy may be assessed by the absence of disease progression as indicated by lack of hospitalization or the need for additional medical interventions. Methods for measuring these indicators are well-known to professionals in the field.

A typical starting dose in clinical trials ranges from 6 to 12.5 mg/m², administered intravenously, generally on a schedule such as once every three or four weeks. In some instances, the effective dose may be the maximum tolerated dose.

The administration dose should be tailored to the individual's requirements. It is known that in humans, spironolactone is administered at doses of 25 to 50 mg daily for cardiac failure and 100 to 400 mg daily for hyperaldosteronism. Studies have explored the use of spironolactone within a range of 25 to 400 mg regarding its potential adverse effects. For cancer treatment, the dose can range from 25 to 400 mg daily. As an example, spironolactone dose concentrations utilized may range from 5 to 25 μM in mice (i.p.) and 20 to 200 mg daily (oral) in clinical settings for humans.

The term “treat” encompasses both therapeutic and prophylactic treatments, aiming to decrease, suppress, attenuate, diminish, arrest, or stabilize the development or progression of a disease, reduce the severity of the disease, or improve symptoms associated with the disease.

The pharmaceutical compositions may be included in a container, pack, or dispenser alongside instructions for administration.

The compositions of the present invention can form salts, potentially more than one salt per molecule, including mono-, di-, tri-, etc. All these forms are contemplated within the scope of the invention.

“Pharmaceutically acceptable salts” refers to derivatives of the compounds of the present invention where the parent compound is modified by forming acid or base salts thereof. Examples include mineral or organic acid salts of basic residues such as amines, and alkali or organic salts of acidic residues like carboxylic acids. The pharmaceutically acceptable salts include conventional non-toxic salts or the quaternary ammonium salts of the parent compound formed from non-toxic inorganic or organic acids, for example, but not limited to, those derived from a variety of acids as listed.

The term “selectively” implies a preference for occurrence in one population over another, typically relating to different cell populations, where, for example, a compound of the present invention acts more frequently or effectively on cancerous cells than on normal cells.

The administration routes for the compositions or pharmaceutically acceptable salts or solvates thereof include oral, nasal, transdermal, pulmonary, inhalational, buccal, sublingual, intraperitoneal, subcutaneous, intramuscular, intravenous, rectal, intrapleural, intrathecal, and parenteral routes. In one embodiment, the compound is administered orally. Suitable administration routes can be determined based on advantages recognized by one skilled in the art.

Formulation and administration techniques for the compounds disclosed are well-documented and can be found in standard references like Remington: The Science and Practice of Pharmacy. The compounds, including pharmaceutically acceptable salts, can be used in pharmaceutical preparations with appropriate carriers or diluents, with dosages adjusted to provide the desired active quantity within the range discussed.

Techniques for formulation and administration of compounds disclosed herein are well-documented and available in standard references such as “Remington: The Science and Practice of Pharmacy, 19th edition”. These compounds, including their pharmaceutically acceptable salts, are suitable for use in pharmaceutical preparations with the appropriate carriers or diluents. The compositions will contain amounts sufficient to provide the desired dose within the ranges described herein.

All percentages and ratios used herein, unless otherwise specified, are by weight. The features and advantages of the present invention are apparent from the provided examples, which illustrate various components and methodologies. These examples should not be construed as limiting the scope of the invention. Based on this disclosure, those skilled in the art can identify other components and methodologies suitable for practicing the invention.

A “subject in need thereof” includes individuals suffering from a precancerous condition or cancer. Subjects encompass mammals such as humans, primates, dogs, cats, mice, rats, horses, and livestock. Preferably, the subject is a human diagnosed with, showing symptoms of, or at risk of developing cancer or a precancerous condition.

A subject may have cancer that is refractory or resistant, meaning the cancer does not respond or stops responding to treatment. This resistance may be inherent at the onset of treatment or may develop over time. In some cases, the subject may have experienced cancer recurrence after a period of remission or may have exhausted all known effective cancer treatments.

In some instances, a subject may present with secondary cancer, which arises as a consequence of previous cancer treatments, such as chemotherapy or radiation therapy.

Cancer symptoms vary widely based on the cancer's location, size, and its impact on surrounding organs or tissues. If the cancer has metastasized, symptoms may manifest in different parts of the body.

Treatment success is often measured by a reduction in tumor size, referred to as “tumor regression”. Ideally, post-treatment, the tumor size is reduced by a significant percentage relative to its size prior to treatment.

Similarly, a successful treatment can lead to a reduction in the number and size of tumors. This can be quantified by a significant percentage decrease relative to the condition before treatment.

Effective cancer treatment may also result in a reduced number of metastatic lesions in tissues or organs distant from the primary tumor site, with a significant decrease in the number of lesions following treatment.

An increase in the average survival time of treated subjects compared to those receiving standard care or a placebo can signify effective treatment. This increase in survival time is preferably more than a certain number of days, highlighting the treatment's efficacy.

Moreover, treating cancer effectively can lead to an increase in the average survival time compared to subjects receiving either no treatment or a different monotherapy.

A successful cancer treatment may also result in a significant decrease in the population's mortality rate, indicating the treatment's effectiveness compared to no treatment or different treatment options.

An effective treatment for cancer can lead to a reduction in the tumor growth rate. Ideally, the rate of tumor growth is decreased by a significant percentage following treatment, showcasing the treatment's ability to slow down or halt tumor progression.

Additionally, successful treatment can result in decreased tumor regrowth. After the initial treatment phase, the likelihood or extent of tumor recurrence is significantly reduced, further demonstrating the treatment's long-term effectiveness.

In the context of treating or preventing cell proliferative disorders, an impactful outcome includes a reduction in the rate of cellular proliferation. Following treatment, this rate is substantially decreased, indicating a slowdown in the proliferation of potentially cancerous or abnormal cells.

Moreover, treating or preventing such disorders can lead to a reduction in the proportion of proliferating cells. This outcome, often measured by the mitotic index or similar metrics, signifies a beneficial shift towards a lesser degree of uncontrolled cell division.

A notable treatment effect is also seen in the decrease in size of areas or zones of cellular proliferation. After treatment, these areas, indicative of aggressive or uncontrolled cell growth, are significantly reduced, reflecting the treatment's efficacy in targeting and limiting cancerous growth.

Another positive outcome is the reduction in the number or proportion of cells exhibiting abnormal appearance or morphology. This change, observable under microscopic examination, indicates a normalization of cellular structures potentially affected by cancerous transformations.

Administering a composition of the present invention to cells or subjects in need thereof can result in modulation of protein methyltransferase activity. This effect, whether stimulation or inhibition, is selectively targeted towards cancer cells, leaving normal cells largely unaffected.

Treating cancer or cell proliferative disorders can induce cell death, a critical component of effective cancer therapy. Preferably, the treatment results in a significant reduction in the number of cells within a treated population, demonstrating the treatment's capability to eradicate cancerous cells effectively. LP-184 is a member of the acylfulvene family of compounds, which are known to induce DNA lesions. These lesions are typically repaired through the Transcription-Coupled Nucleotide Excision Repair (TC-NER) pathway.

Contacting a cell with a composition of the present invention can activate cell death selectively in cancer cells. This selective induction is crucial for minimizing harm to normal cells while effectively combating cancerous growths.

The method of treating or preventing cancer involves administering a composition of the present invention, leading to one or more beneficial outcomes. These may include the prevention of cancer cell proliferation, induction of cell senescence, or promotion of tumor cell differentiation, all while maintaining a favorable therapeutic index.

The term “kit” refers to a package that may contain one or more components of the treatment regimen, allowing for dosing flexibility. Components can be administered simultaneously or separately, with adjustments made for timing and dosage to achieve optimal treatment outcomes.

The “predetermined level” serves as a threshold: if ACTB levels in a patient are above or below this threshold, it can guide the healthcare provider in deciding whether to initiate, continue, or adjust the treatment with acylfulvene.

For detailed descriptions of techniques and methodologies, one skilled in the art may refer to general reference texts. These resources provide a foundation for making or using aspects of the invention, ensuring that practices are grounded in established scientific knowledge.

### EXAMPLES

In order that the disclosure disclosed herein may be more efficiently understood, examples are provided below. It should be understood that these examples are for illustrative purposes only and are not to be construed as limiting the disclosure in any manner.

### Example 1

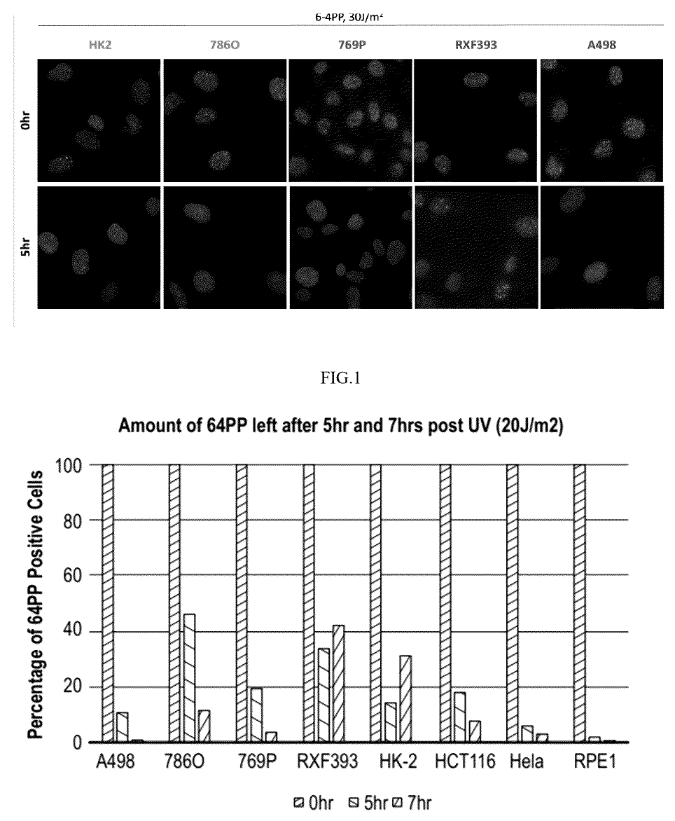

FIG. 1 demonstrates the effects of LP-184 on various renal cell lines, including both epithelial and carcinoma types. The assay assesses the ability of the cells to repair 6-4 photoproducts, showing a spectrum of responses: non-malignant HK2 cells display typical non-malignant behavior; 786O and 769P cell lines show low sensitivity; and clear cell renal carcinoma cell lines such as A498 and RXF393 exhibit high sensitivity. Following UV irradiation, which induces 6-4 photoproducts (6-4PP), it was noted that almost all kidney cancer cell lines, along with a non-malignant kidney epithelial cell line, exhibited varying levels of nucleotide excision repair (NER) deficiency, as depicted in the figure. By contrast, the RPE1 cell lines acted as a negative control, showcasing a distinct reaction to the treatment.

### Example 2

FIG. 2 illustrates the extent of nucleotide excision repair (NER) deficiency among renal cancer and control cells. Cells with 6-4 photoproducts (6-4PPs) were significantly eliminated over periods of 5 and 7 hours. 6-4PPs are specific DNA lesions that result from UV radiation linking adjacent pyrimidine bases (thymine or cytosine) in the DNA. Such lesions can interfere with cellular functions and impede DNA replication and transcription if left unrepaired. The findings indicate that all kidney cancer cell lines and a kidney epithelial non-malignant cell line harbored NER deficiency to some extent, which can be targeted by LP-184.

### Example 3

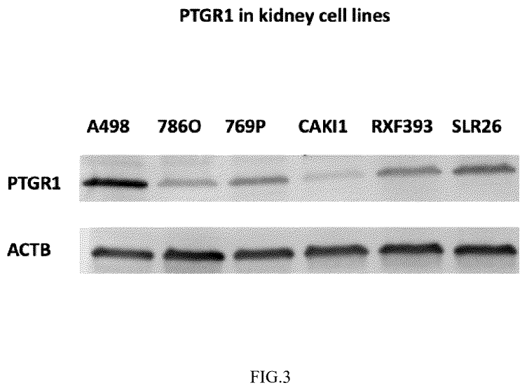

FIG. 3 presents a western blot analysis conducted on the previously mentioned cell lines (non-malignant HK2 cells showing typical behavior; 786O and 769P lines exhibiting low sensitivity; and clear cell renal carcinoma cell lines such as A498 and RXF393), investigating PTGR1 and ACTB gene expression. The blot indicates that cells expressing PTGR1 are more sensitive to LP-184 treatment.

### Example 4

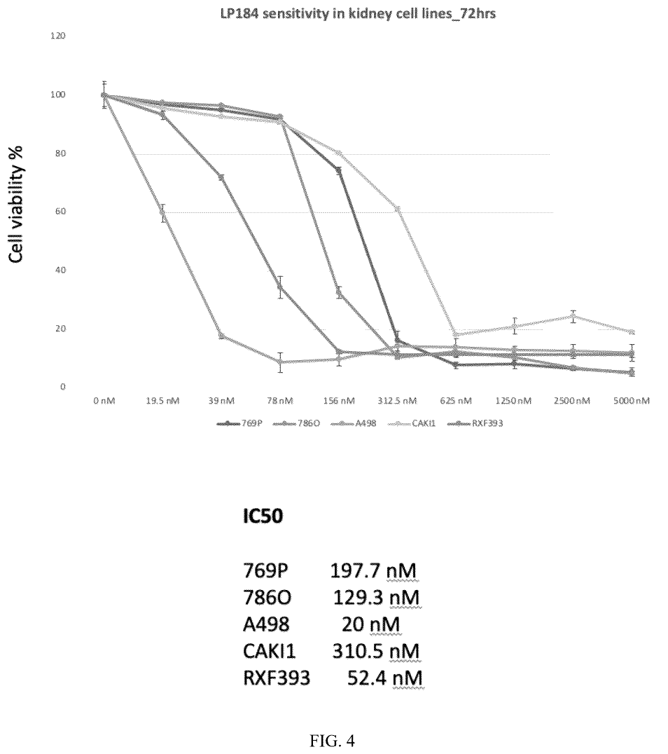

FIG. 4 details the sensitivity of various cell lines to LP-184, showing cell death over 72 hours. Sensitivity, gauged by IC50 data, reflects how effectively a substance can inhibit a particular biological or biochemical function. Here, the IC50 data reveals the rate at which cells die over a 72-hour period, a critical metric for evaluating the potency of a compound. This IC50 value, indicating the concentration needed to reduce cell viability by 50%, is key to understanding the compound's efficacy and the temporal response of the cells to the substance. Such analyses are vital for new drug development and therapeutic approaches, offering important insights into how cells react to different compounds over time.

Additional IC50 data is presented below:

| Cell Line | Tissue | LP-184 IC50 (nM) |
|---|---|---|
| ACHN | Kidney | 222 |
| SN12C | Kidney | 349 |
| TK10 | Kidney | 314 |
| UO31 | Kidney | 178 |

Additional IC50 data is presented below:

| | | LP-184 | PTGR1 (RNASeq Expression in |
|---|---|---|---|
| Cell Line | Tissue | IC50 (nM) | Log2(FPKM + 1)) |
| X7860 | Kidney | 331 | 2.9 |
| A498 | Kidney | 179 | 3.88 |
| ACHN | Kidney | 222 | 3.03 |
| CAK11 | Kidney | 282 | 2.93 |
| RXF393 | Kidney | 152 | 4.21 |
| SN12C | Kidney | 349 | 3.17 |
| TK10 | Kidney | 314 | 3.89 |
| UO31 | Kidney | 178 | 4.01 |

This example shows the sensitivity of various cell lines to LP-284, showing cell death over 72 hours. Sensitivity, gauged by IC50 data, reflects how effectively a substance can inhibit a particular biological or biochemical function. Here, the IC50 data reveals the rate at which cells die over a 72-hour period, a critical metric for evaluating the potency of a compound. This IC50 value, indicating the concentration needed to reduce cell viability by 50%, is key to understanding the compound's efficacy and the temporal response of the cells to the substance. Such analyses are vital for new drug development and therapeutic approaches, offering important insights into how cells react to different compounds over time. Such data is shown below:

| | | LP-184 | LP-284 | |
|---|---|---|---|---|
| Cell Line | Tissue | IC50 (nM) | IC50 (nM) | PTGR1 |
| X7860 | Kidney | 331 | 2236 | 2.9 |
| A498 | Kidney | 179 | 1350 | 3.88 |
| ACHN | Kidney | 222 | 1130 | 3.03 |
| CAK11 | Kidney | 282 | 1910 | 2.93 |
| RXF393 | Kidney | 152 | 328 | 4.21 |
| SN12C | Kidney | 349 | 2620 | 3.17 |
| TK10 | Kidney | 314 | 2010 | 3.89 |
| UO31 | Kidney | 178 | 254 | 4.01 |

While several exemplary aspects and embodiments have been provided, those skilled in the art will recognize possible modifications, combinations, permutations, and variations. Therefore, the appended claims and any claims subsequently introduced are to be interpreted to encompass all such modifications, combinations, permutations, and variations within their true spirit and scope.

1. A method of treating renal cell carcinoma in a subject in need thereof, comprising administering to the subject an effective amount of hydroxyureamethyl acylfulvene.

2. The method of claim 1, wherein the hydroxyureamethyl acylfulvene is (−)-hydroxyureamethyl acylfulvene.

3. The method of claim 1, wherein the hydroxyureamethyl acylfulvene is (+)-hydroxyureamethyl acylfulvene.

4. The method of claim 1, wherein administration is intravenous (IV) or intraperitoneal (IP).

5. A method of treating renal cell carcinoma in a subject in need thereof, comprising administering to the subject acylfulvene or a pharmaceutically acceptable salt thereof.

6. The method of claim 1, wherein the renal cell carcinoma is localized to the kidney.

7. The method of claim 1, wherein the renal cell carcinoma is metastatic.

8. The method of claim 1, wherein the renal cell carcinoma is selected from clear cell renal cell carcinoma, papillary renal cell carcinoma, chromophobe renal cell carcinoma, collecting duct carcinoma, and medullary carcinoma.

9. The method of claim 1, wherein the subject is a non-human mammal.

10. The method of claim 1, wherein the subject is a human.

11. The method of claim 1, further comprising administering at least one additional therapeutic agent.

12. The method of claim 11, wherein the additional therapeutic agent is selected from camptothecin derivatives, paclitaxel, docetaxel, epothilone B, 5-fluorouracil, gemcitabine, oxaliplatin, cisplatin, carboplatin, melphalan, dacarbazine, temozolomide, doxorubicin, imatinib, erlotinib, bevacizumab, cetuximab, and a Raf kinase inhibitor.

13. The method of claim 1, wherein the hydroxyureamethyl acylfulvene is administered in a dosage range of 0.1 to 100 mg/kg of body weight per day.

14. The method of claim 1, wherein the hydroxyureamethyl acylfulvene is administered at 6 to 12.5 mg/m² IV on a three- or four-week cycle.

15. The method of claim 1, wherein treatment reduces tumor size or slows tumor progression.

16. The method of claim 1, wherein the subject's tumor over-expresses PTGR1.

17. The method of claim 16, wherein PTGR1 over-expression is determined by immunohistochemistry or RNA-sequencing.

18. The method of claim 1, wherein the subject is selected by identifying nucleotide excision repair deficiency in a tumor sample using immunofluorescent detection of photoproducts.

19. The method of claim 1, wherein the hydroxyureamethyl acylfulvene is administered as monotherapy.

20. A pharmaceutical composition for intravenous administration comprising (−)-hydroxyureamethyl acylfulvene and a pharmaceutically acceptable carrier, for use in treating clear cell renal cell carcinoma in a human subject selected by (i) elevated PTGR1 expression in a tumor sample as determined by Western blot or RNA sequencing and (ii) nucleotide excision repair deficiency as determined by persistence of photoproducts relative to non-malignant kidney epithelial cells.
